(19) United States
(12) Patent Application Publication
(10) Pub. No.: US 2009/0118722 A1
(43) Pub. Date: May 7, 2009

(54) METHOD AND APPARATUS FOR COOLING SUBCUTANEOUS LIPID-RICH CELLS OR TISSUE

(76) Inventors: Edward A. Ebbers, San Carlos, CA (US); Mitchell E. Levinson, Pleasanton, CA (US); Jessica Preciado, Alameda, CA (US); Nathan R. Every, Seattle, WA (US); John W. Allison, Los Altos, CA (US)

Correspondence Address: Zeltiq Aesthetics, Inc.;Perkins Coie LLP, P.O. BOX 1247, SEATTLE, WA, 98111-1247, US

(21) Appl. No.: 11/933,066

(22) Filed: Oct. 31, 2007

Related U.S. Application Data
(60) Provisional application No. 60/855,784, filed on Oct. 31, 2006.

(51) Int. Cl. A61B 18/02

(52) U.S. Cl. 606/21

(57) ABSTRACT

A system for reducing subcutaneous lipid-rich cells or tissue of a subject is disclosed. The system may include a fluid supply, a probe in fluid communication with the fluid supply, and a coolant circulated between the fluid supply and the probe. The probe may be configured to be inserted into the subject to be at least proximate to the subcutaneous lipid rich cells. The coolant may be at a temperature such that the subcutaneous lipid-rich cells or tissue proximate to the inserted probe are cooled.

### CROSS-REFERENCE TO RELATED APPLICATIONS

This application claims the benefit of U.S. Provisional Application No. 60/855,784, filed Oct. 31, 2006, and entitled “METHOD AND APPARATUS FOR COOLING SUBCUTANEOUS LIPID-RICH CELLS OR TISSUE,” the entire disclosure of which is incorporated herein by reference.

### TECHNICAL FIELD

The present application relates to cooling apparatuses, systems, and methods for selectively affecting subcutaneous lipid-rich cells or tissue, and more particularly, a method and system having one or more probes for inserting into a subject directly to cool and/or heat subcutaneous lipid-rich cells or tissue of the subject.

### BACKGROUND

Excess body fat, or adipose tissue, may be present in various locations of the body, including, for example, the thigh, buttocks, abdomen, knees, back, face, arms, and other areas. Excess adipose tissue can detract from personal appearance and athletic performance. Moreover, excess adipose tissue is thought to magnify the unattractive appearance of cellulite, which forms when subcutaneous fat protrudes into the dermis and creates dimples where the skin is attached to underlying structural fibrous strands. Cellulite and excessive amounts of adipose tissue are often considered to be unappealing. Moreover, significant health risks may be associated with higher amounts of excess body fat. An effective way of controlling or removing excess body fat therefore is needed.

Liposuction is a method for selectively removing adipose tissue to “sculpt” a person's body. Liposuction typically is performed by plastic surgeons or dermatologists using specialized surgical equipment that invasively removes subcutaneous adipose tissue via suction. One drawback of liposuction is that it is a surgical procedure, and the recovery may be painful and lengthy. Moreover, the procedure typically requires the injection of tumescent anesthetics, which is often associated with temporary bruising. Liposuction can also have serious and occasionally even fatal complications. In addition, the cost for liposuction is usually substantial. Other emerging techniques for removal of subcutaneous adipose tissue include mesotherapy, laser-assisted liposuction, and high intensity focused ultrasound.

Conventional non-invasive treatments for removing excess body fat typically include topical agents, weight-loss drugs, regular exercise, dieting, or a combination of these treatments. One drawback of these treatments is that they may not be effective or even possible under certain circumstances. For example, when a person is physically injured or ill, regular exercise may not be an option. Similarly, weight-loss drugs or topical agents are not an option when they cause an allergic or negative reaction. Furthermore, fat loss in selective areas of a person's body cannot be achieved using general or systemic weight-loss methods.

Other non-invasive treatment methods include applying heat to a zone of subcutaneous lipid-rich cells. U.S. Pat. No. 5,948,011 discloses altering subcutaneous body fat and/or collagen by heating the subcutaneous fat layer with radiant energy while cooling the surface of the skin. The applied heat denatures fibrous septae made of collagen tissue and may destroy fat cells below the skin, and the cooling protects the epidermis from thermal damage. This method is less invasive than liposuction, but it still may cause thermal damage to adjacent tissue, and can also be painful and unpredictable.

Additional methods and devices to reduce subcutaneous adipose tissue are disclosed in U.S. Patent Publication Nos. 2003/0220674 and 2005/0251120, the entire disclosures of which are incorporated herein by reference. Although the methods and devices disclosed in these publications are promising, several improvements for enhancing the implementation of these methods and devices would be desirable.

### BRIEF DESCRIPTION OF THE DRAWINGS

FIG. 1 is an isometric view of a system for cooling subcutaneous lipid-rich cells or tissue in accordance with an embodiment of the invention.

FIG. 2A is a top view of a cooling device having a plurality of probes in accordance with an embodiment of the invention.

FIG. 2B is a side cross-sectional view of a cooling device having an evacuation chamber in accordance with an embodiment of the invention.

FIGS. 3A and 3B are side elevation views partially illustrating an embodiment of a probe of FIG. 2A. FIGS. 3C and 3D are side elevation views partially illustrating another embodiment of the probe. FIG. 3E is a perspective view partially illustrating yet another embodiment of a probe of FIG. 2A.

FIG. 4 is a side cross-sectional view illustrating a needle portion of the probe of FIG. 2A in accordance with an embodiment of the invention.

FIG. 5 is a side cross-sectional view illustrating a needle portion of the probe of FIG. 2A in accordance with another embodiment of the invention.

FIGS. 6A-B are top views illustrating the probe of FIG. 2A operated in accordance with another embodiment of the invention.

FIG. 7 is a block diagram showing computing system software modules for cooling subcutaneous lipid-rich cells or tissue.

FIG. 8 is a flowchart showing a method of treatment planning suitable for execution in the processor of FIG. 7.

### DETAILED DESCRIPTION

### A. Overview

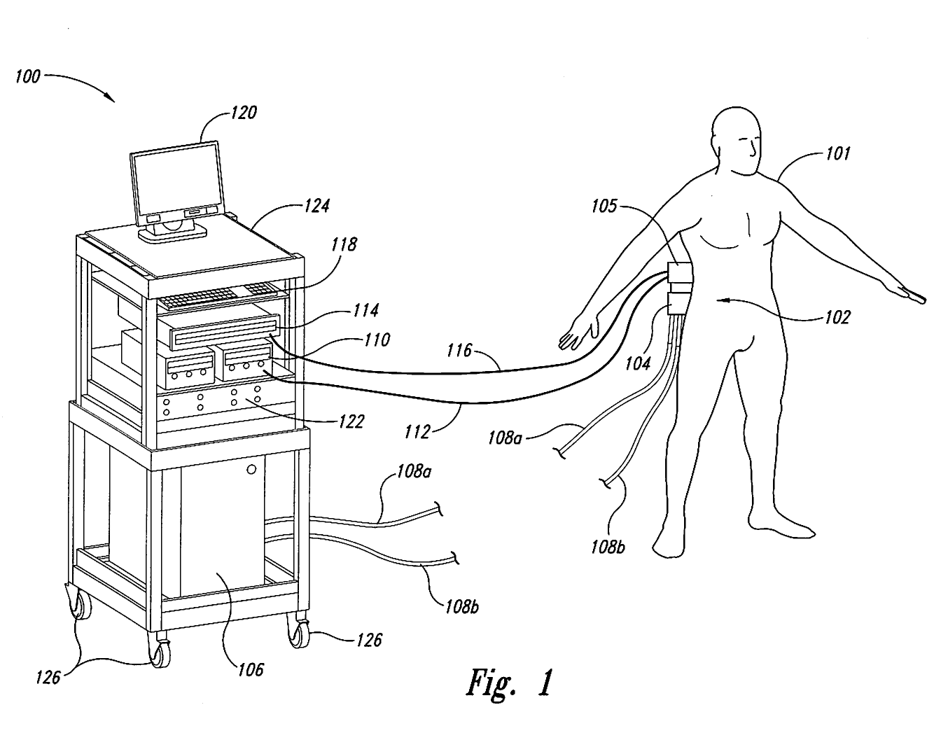

The present disclosure describes devices, systems, and methods for cooling subcutaneous lipid-rich cells or tissue. It will be appreciated that several of the details set forth below are provided to describe the following embodiments in a manner sufficient to enable a person skilled in the relevant art to make and use the disclosed embodiments. Several of the details and advantages described below, however, may not be necessary to practice certain embodiments of the invention. Additionally, the invention can include other embodiments that are within the scope of the claims but are not described in detail with respect to FIGS. 1-8.

### B. System for Selectively Reducing Lipid-Rich Cells or Tissue

FIG. 1 is an isometric view of a system 100 for cooling subcutaneous lipid-rich cells or tissue of a subject 101 in accordance with an embodiment of the invention. The system 100 can include a treatment device 104 placed at an abdominal area 102 of the subject 101 or another suitable area for cooling the subcutaneous lipid-rich cells or tissue of the subject 101.

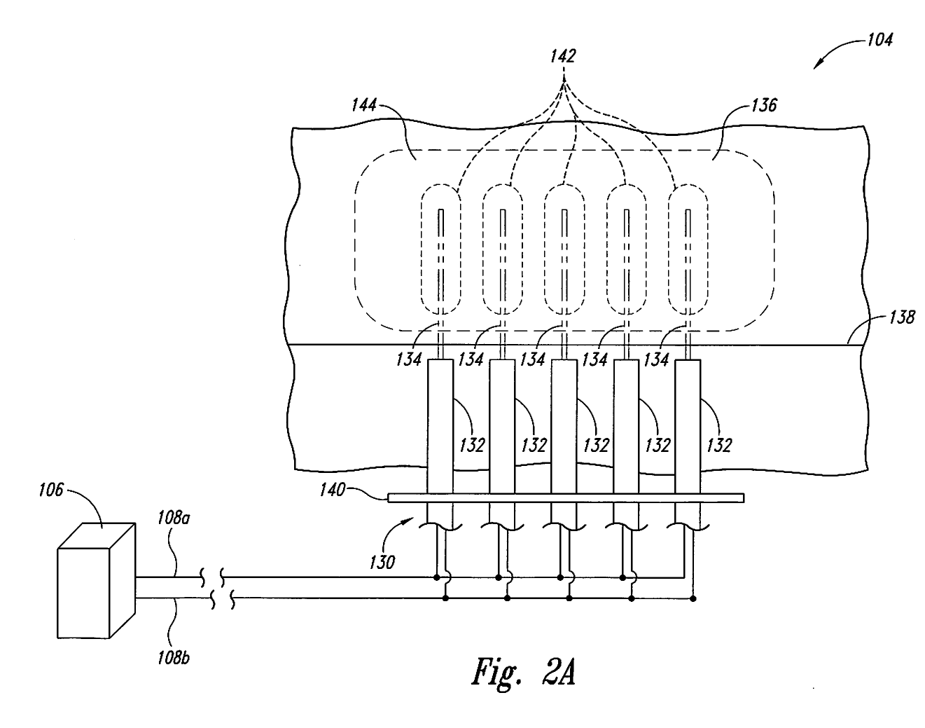

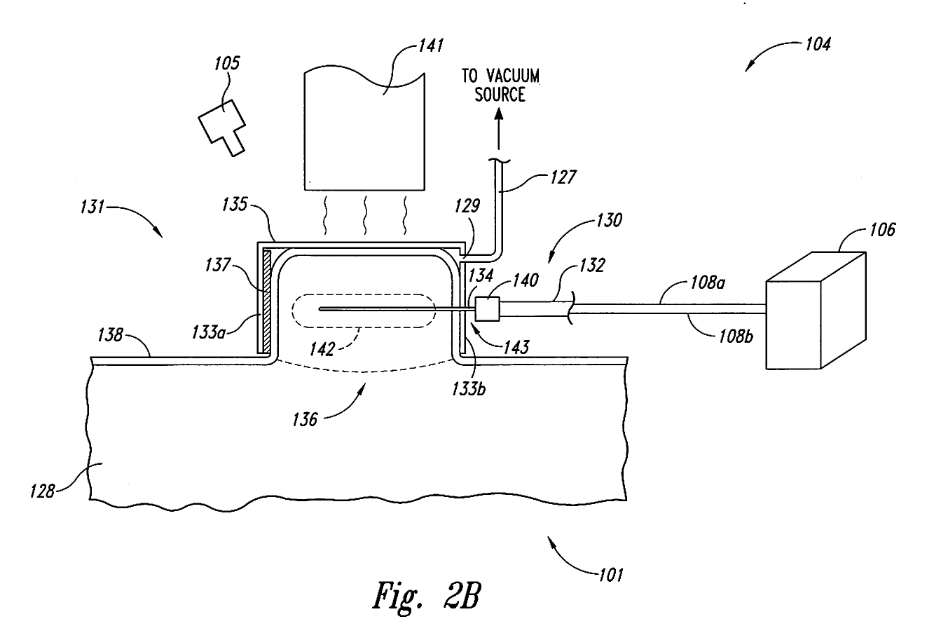

The treatment device 104 may include one or more probes (shown in FIGS. 2-6) for inserting into the subject 101 and directly cooling and/or heating the subcutaneous lipid-rich cells or tissue of the subject 101. The treatment device 104 may also include external non-invasive cooling units for pre-cooling the subcutaneous lipid-rich cells or tissue and/or numbing the skin of the subject 101 proximate to the subcutaneous lipid-rich cells or tissue. Such non-invasive cooling units may also be used in conjunction with the probes to reduce subcutaneous adipose tissue, for example, as disclosed in U.S. Patent Publication Nos. 2003/0220674 and 2005/0251120 and other references disclosed herein. For example, the non-invasive cooling units may include a device having cooling elements such as those disclosed in U.S. patent application Ser. No. 11/359,092, entitled “Cooling Device for Removing Heat From Subcutaneous Lipid-Rich Cells,” filed Feb. 22, 2006, by Ting et al., the entire disclosure of which is incorporated herein by reference. In another example, the non-invasive cooling units may include other external cooling components including, for example, ice packs and evaporative materials that may be applied to the skin of the subject 101. For instance, devices and methods described in U.S. patent application Ser. No. 11/435,502, entitled “Method and Apparatus for Removing Heat from Subcutaneous Lipid-Rich Cells Including a Coolant Having a Phase Transition Temperature” by Levinson, the entirety of which is incorporated herein by reference, may be used with the present invention. Other devices, features and methods as described in U.S. patent application Ser. No. 11/528,189, entitled “Cooling Device with Flexible Sensors”, filed Sep. 26, 2006 to Levinson et al., and U.S. patent application Ser. No. 11/528,225 entitled “Cooling Device Having a Plurality of Controllable Cooling Elements to Provide a Predetermined Cooling Profile”, filed Sep. 26, 2006 to Levinson et al, the entirety of each which is incorporated herein by reference, may also be used with the present invention. Various embodiments of the treatment device 104 are described in more detail below with reference to FIGS. 2-6.

The system 100 may further include a fluid supply 106 and fluid lines 108a-b connecting the treatment device 104 to the fluid supply 106. The fluid supply 106 may generate and circulate a fluid to the treatment device 104 via the fluid lines 108a-b. Examples of the circulating fluid include water, ethylene glycol, synthetic heat transfer fluid, oil, refrigerant, liquid nitrogen, liquid argon, and any other suitable heat-conducting fluid. The fluid lines 108a-b may be hoses or other conduits constructed from polyethylene, polyvinyl chloride, polyurethane, or other materials that can accommodate the particular circulating fluid. The fluid supply 106 may be fluidly connected to a refrigeration unit, a cooling tower, a thermoelectric chiller, an ambient vaporizer, or any other device capable of delivering a coolant. In a particular embodiment, the fluid supply 106 may include a liquid nitrogen container that can store and circulate liquid nitrogen as a critical liquid without vaporization. One suitable liquid nitrogen container is the Critical N₂ generator manufactured by Endocare, Inc., of Irvine, Calif.

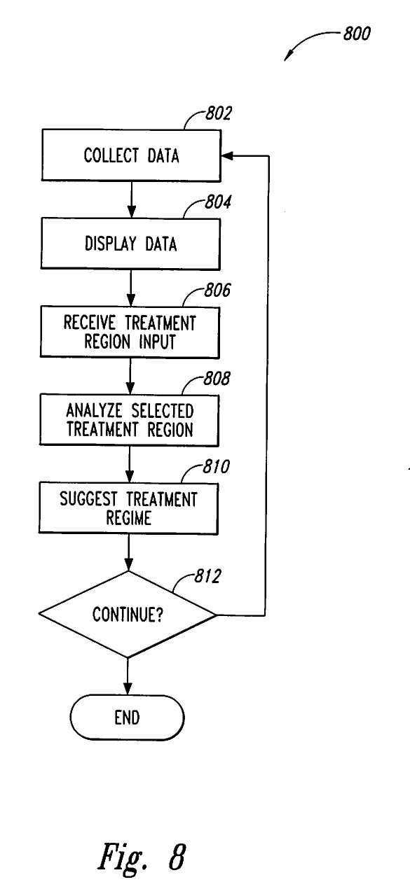

The system 100 may further include sensors for monitoring a treatment process. For example, the system 100 may include a detector 105 that includes, for example, a temperature sensor, a pressure sensor, an ultrasound sensor, a computed tomography scanner, a radioscopy scanner, an X-ray machine, and/or an MRI scanner. The detector 105 may be configured for detecting process parameters (e.g., temperature, pressure, blood flow, tissue density and other physiological parameters) and/or for facilitating the placement of the treatment device 104, as described in more detail below with reference to FIG. 8. The detector 105 may be electrically coupled to a power supply 110 via a power cable 112 and to a processing unit 114 via a signal cable 116 or wireless means (radio frequency, infrared, etc.).

The processing unit 114 can control process parameters from the detector 105 and adjust the treatment process based on the monitored process parameters. The processing unit 114 may also be in electrical communication with an input device 118, an output device 120, and/or a control panel 122. The processing unit 114 may include any processor, Programmable Logic Controller, Distributed Control System, and the like. The input device 118 may include a keyboard, a mouse, a touch screen, a push button, a switch, a potentiometer, and any other devices suitable for accepting user input. The output device 120 may include a display screen, a printer, a medium reader, an audio device, and any other devices suitable for providing user feedback. The control panel 122 may include audio devices and one or more visual displays having, e.g., indicator lights, numerical displays, etc. In the embodiment shown in FIG. 1, the processing unit 114, power supply 110, control panel 122, fluid supply 106, input device 118, and output device 120 are carried by a rack 124 with wheels 126 for portability. In another embodiment, the various components may be fixedly installed at a treatment site. Features, devices and methods described in U.S. patent application Ser. No. 11/777,992 entitled “System for Removing Heat from Lipid-Rich Regions” to Levinson et al., the entirety of which is incorporated herein by reference, may be used with the present invention.

In operation, an operator may place the treatment device 104 proximate to the subcutaneous lipid-rich cells or tissue of a desired treatment region and then cool the treatment device 104 to affect the subcutaneous lipid-rich cells or tissue in the treatment region. For example, the operator may turn on the fluid supply 106 to circulate a coolant at a given temperature (e.g., about 5° C., about 0° C., about −5° C., or about −10° C.) to cool the treatment device 104, which in turn conducts heat away from the subcutaneous lipid-rich cells or tissue in the treatment region. Other cooling techniques, such as evaporative cooling, may be used in lieu of or in addition to the treatment device 104. The treatment device 104 may be controlled to cool, but not freeze, the subcutaneous lipid-rich cells or tissue. In other embodiments, the treatment device 104 can selectively freeze the subcutaneous lipid-rich cells or tissue, or freeze the subcutaneous lipid-rich cells or tissue in the treatment region and affect the cells adjacent to the treatment region. The present invention is directed to cooling subcutaneous lipid-rich cells or tissue without freezing, freezing alone, or freezing and cooling adjacent subcutaneous lipid-rich cells or tissue. The treatment device 104 may include one or more probes, each of which may be dedicated in any combination for freezing and/or cooling without freezing, and may affect any combination of freezing and/or cooling without freezing. Furthermore, the probe(s) may be employed to effect a desired volume of treatment region.

During cooling, the skin and/or other tissues of the subject 101 may be protected by applying heat to the skin surface. For example, a warm fluid (e.g., a saline solution or other biocompatible solution) may be applied external to the skin of the subject 101 during treatment. The warm fluid can maintain a select temperature of the skin of the subject 101 and thus prevent the skin from overcooling. In other examples, the operator may apply heat to the skin surface using resistive heating elements, radiofrequency energy, ultraviolet light, ultrasound, microwave, or other suitable heating techniques. In one particular embodiment, capacitively coupled radiofrequency is used to apply heat to the skin surface.

By cooling the subcutaneous tissues to a temperature lower than 37° C., subcutaneous lipid-rich cells or tissue may be selectively affected. In general, surrounding tissues of the subject 101 (e.g., the dermis) typically have lower amounts of unsaturated fatty acids compared to the underlying lipid-rich cells or tissue that form the subcutaneous tissues. Because non-lipid-rich cells or tissue usually can withstand colder temperatures better than lipid-rich cells or tissue, the subcutaneous lipid-rich cells or tissue may selectively be affected while maintaining the non-lipid-rich cells or tissue in the surrounding tissues. The lipid-rich cells or tissue may be affected by disrupting, shrinking, disabling, destroying, removing, killing, or otherwise being altered. Without being bound by theory, cooling is believed to injure lipid-rich cells or tissue, inducing apoptosis or necrosis, resulting in cell destruction and subsequent resorption through the body's natural wound-healing mechanisms.

After cooling the subcutaneous lipid-rich cells or tissue, the operator optionally may stop cooling such cells or even apply heat to the cooled cells to promote reperfusion injury of these cells. For example, the operator may stop the fluid supply 106 from circulating the coolant through the treatment device 104. Further, the operator optionally may then apply a heating fluid to the treatment device 104. In one embodiment, the heating fluid may be circulated through the treatment device 104. The heating fluid may include water, ethylene glycol, synthetic heat transfer fluid, oil, and any other suitable heat-conducting fluids. In other embodiments, the heating fluid (e.g., a saline solution or other biocompatible solution) may be released into the subject 101 so that the warm fluid warms the subcutaneous lipid-rich cells or tissue. In further embodiments, the operator may warm the cooled treatment region using resistive heating elements, radiofrequency energy, ultraviolet light, ultrasound, microwave, or other suitable heating techniques. Accordingly, the present invention contemplates application of temperatures ranging from about −200° C. or colder to about 42° C. or warmer, depending on the particular treatment regime selected and the various embodiments and other devices therein employed. For example, cryoablation may be carried out at a temperature around −75° C.

According to further embodiments, after the subcutaneous lipid-rich cells or tissue are warmed to a desired temperature (e.g., 20° C.), the operator may stop applying the heating fluid and, optionally, switch back to circulating the coolant through the treatment device 104. During either cooling or warming, once a desired temperature is achieved, the temperature of the region may be maintained for a predetermined period of time. In certain embodiments, this cooling/warming process may be repeated until a desired reduction in lipid-rich cells or tissue in the treatment region is achieved over a period of time or for a desired cooling/warming profile. In another embodiment, the treatment device 104 may be applied to a different portion of the skin as described above to selectively affect lipid-rich cells or tissue in a different subcutaneous target region. Further, the treatment may be reapplied to a given treatment region until a desired reduction in lipid-rich cells or tissue in that treatment region is achieved.

During treatment, the operator may monitor the treatment process using the detector 105 and the processing unit 114. For example, the detector 105 may measure a process parameter (e.g., a temperature, chemical, electrical, or mechanical change in the treatment region, cells adjacent to the treatment region, or on the surface of the skin in proximity to the treatment region), convert the measured parameter into an electrical signal, and transmit the signal to the processing unit 114 to be displayed on the output device 120. The detector 105 may measure a process parameter for the treatment region as well as for other regions of the subject 101. For example, the detector 105 may measure parameters for the skin, other tissues, and/or the organs of the subject 101.

In some embodiments, before treating the subject 101 with the treatment device 104, a tumescent fluid may be injected into or near the target region. Examples of the tumescent fluid include lidocaine, epinephrine, or other suitable tumescent fluids. One expected advantage of injecting a tumescent fluid is that the injected fluid can act as a local anesthetic and can expand the volume of fatty tissue in the treatment region to improve treatment efficacy. The operator can also inject one or more markers into the treatment region to aid the identification of the subcutaneous lipid-rich cells or tissue in the treatment region. For example, the operator can use a biocompatible dye or nanoparticles to define the boundary of the treatment region under MRI imaging, ultrasound, etc.

Several embodiments of the system 100 may reduce the subcutaneous lipid-rich cells or tissue may be reduced generally without any or significant collateral damage to non-lipid-rich cells or tissue in the same region. In general, lipid-rich cells or tissue may be affected at low temperatures that do not affect non-lipid-rich cells or tissue. As a result, lipid-rich cells or tissue, such as those forming the cellulite, may be affected while other cells in the same region are generally not damaged (or are minimally damaged) even though the non-lipid-rich cells or tissue at the surface are subject to even lower temperatures. The treatment device 104 may simultaneously and selectively reduce subcutaneous lipid-rich cells or tissue while providing beneficial effects to the dermis and/or epidermis. These effects may include, for example: fibroplasia, neocollagenesis, collagen contraction, collagen compaction, increase in collagen density, collagen remodeling, and acanthosis (epidermal thickening).

Even though the operation of the system 100 is described in the context of treating subcutaneous lipid-rich cells, the system 100 can also be applied to treat other lipid bearing structures which may or may not include lipid-rich cells. For example, the system 100 may be used to treat lipomas, acne, non-subcutaneous adipose tissue (i.e. “deep” fat), or other types of lipid-bearing structures. Many of these lipid-bearing structures may be treated with non-invasive cooling methods and systems, such as the cooling device disclosed in U.S. patent application Ser. No. 11/359,092; may be treated with various embodiments of system 100; or may be treated by both a non-invasive cooling device and various embodiments of system 100.

### C. Cooling Probes

FIG. 2A is a top view of a specific embodiment of the treatment device 104 suitable for use in the system 100. The treatment device 104 may include one or a plurality of probes 130 arranged into an array and in fluid communication with the fluid supply 106 via the fluid lines 108a-b. Even though five probes 130 are illustrated in FIG. 2A, the treatment device 104 of the present invention, in any of the embodiments contemplated herein, may include any desired number of one or more probes according to the requirement of a treatment region 136.

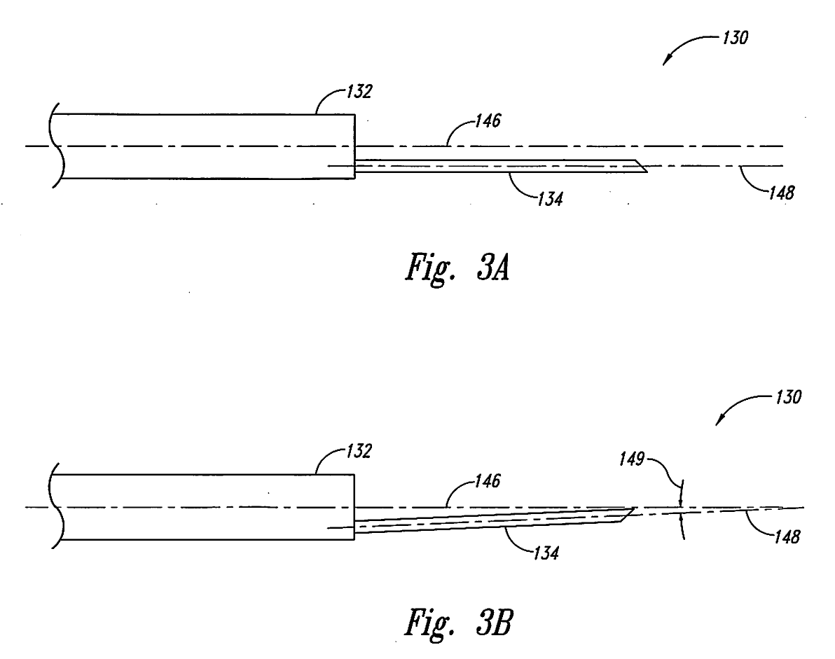

Individual probes 130 may be configured to be inserted into the subcutaneous lipid-rich cells or tissue in the treatment region 136 by piercing the skin 138 of the subject 101 (FIG. 3). In the illustrated embodiment, the probes 130 generally are inserted parallel to each other and at a generally perpendicular angle relative to the surface of the skin 138. In other embodiments, the probes 130 may be inserted at other angles relative to the skin of the subject 101 or to each other. For example, the probes 130 may be inserted at low insertion angles to the skin or at any angle between 0° and 90°.

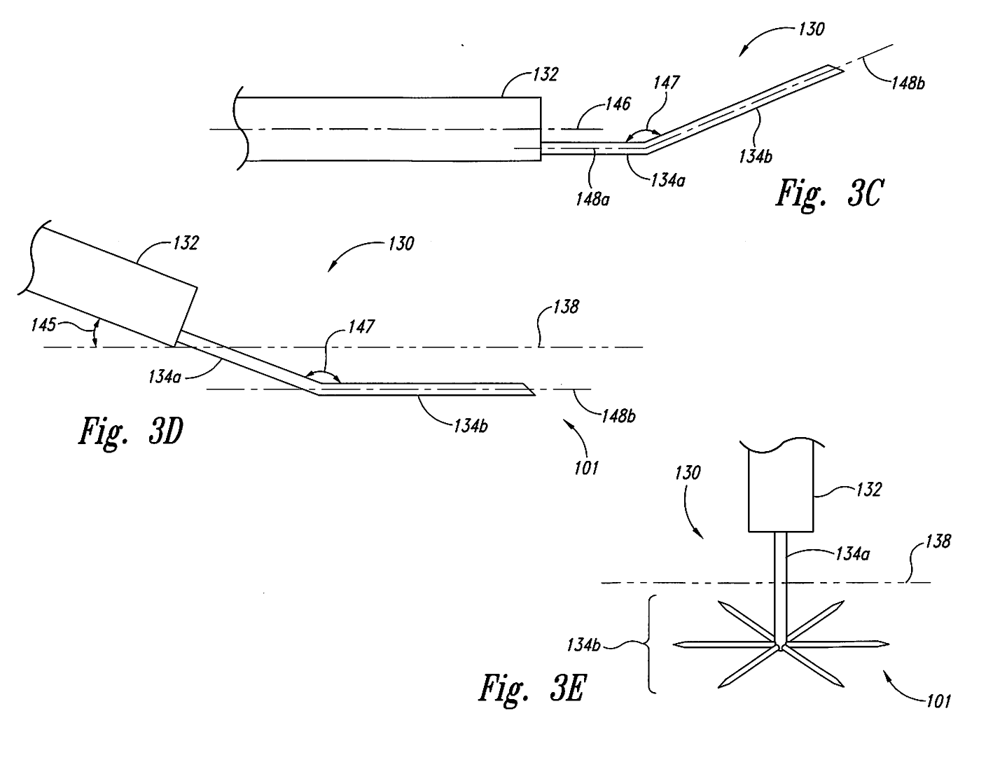

Individual probes 130 may include a cooling element 134 and a base 132. The cooling element 134 may be a thin, rigid needle configured to be inserted into the subject 101, and the base portion 132 may be configured to facilitate such insertion. The cooling element 134 may include fluid passageways in fluid communication with the fluid supply 106, as described in more detail below with reference to FIG. 4. The base portion 132 may include a sleeve surrounding conduits in fluid communication with the internal passageway within the cooling element 134.

Optionally, the treatment device 104 may further include a template 140 for arranging the probes 130 into an array according to the requirement of the treatment region 136. The template 140 may include a substantially rigid plate-like structure having an array of apertures for receiving individual probes 130. One suitable template is a cryoprobe template manufactured by Endocare, Inc., of Irvine, Calif. Alternatively, template 140 may be configured for use with a single probe 130.

In operation, an operator may arrange the probes 130 based on the dimension of the treatment region, and optionally, with the aid of the template 140. Then, the operator may insert the probes 130 into the subcutaneous lipid-rich cells or tissue of the treatment region 136 by piercing the skin 138. During insertion, the operator may use palpation or imaging from the detector 105 to monitor the current position of the probes 130 and adjust the placement of the cooling elements 134 accordingly.

The operator may then use the inserted probes 130 to cool the subcutaneous lipid-rich cells or tissue proximate to the cooling elements 134. For example, the operator may activate the fluid supply 106 to circulate a coolant through the probes 130 via the fluid lines 108a-b. During circulation, the coolant flows from the fluid supply 106 to the probes 130 via the fluid line 108a. The coolant then cools the cooling elements 134 of the probes 130, which in turn conducts heat away from the subcutaneous lipid-rich cells or tissue of the treatment region into the coolant. The coolant with the absorbed heat then returns to the fluid supply 106 via the fluid line 108b.

In the illustrated embodiment, the subcutaneous lipid-rich cells or tissue may be frozen to create treatment zones 142 proximate to the inserted cooling elements 134. The treatment zones 142 may be separated from each other or may be joined to form a contiguous volume of frozen tissue of any desired shape or size. In another embodiment, the probes 130 not only may freeze the cells in the treatment zones 142 but may also affect cells in surrounding areas 144 by creating a temperature gradient in the surrounding areas 144. In a further embodiment, the subcutaneous lipid-rich cells or tissue are cooled without being frozen during treatment. For example, the subcutaneous lipid-rich cells or tissue may be cooled to a temperature lower than a body temperature of the subject 101 without any ice formation in the treatment region. In general, any volume or multiple volumes of a desired shape and size may be created in which such volume or volumes comprise(s) frozen and/or cooled tissue.

Experiments were performed using a cryoprobe system supplied by Endocare, Inc., of Irvine Calif. in a porcine model. During the experiments, a single cryoprobe was placed in a region of subcutaneous adipose tissue of the model. An ice ball was formed in the subcutaneous adipose tissue around the cryoprobe. Subsequent histological examination correlated with ultrasound observation within one hour of treatment confirmed that the frozen lipid-rich tissue had sustained necrotic injury. Concurrently, tissue immediately surrounding the ice ball was observed to sustain a secondary cooling injury. The secondary injury region extended outwardly beyond the outer boundary of the ice ball in a radius having a length between about 70% and about 100% of the ice ball radius. Apoptotic death of a significant portion of the lipid-rich cells in the secondary injury region were observed by subsequent histological examination correlated with ultrasound observation two days after treatment. Histological and ultrasound observations conducted six days, two weeks, four weeks, six weeks, and eleven weeks after treatment revealed a progressive removal of adipocytes via an inflammatory response effected through the necrotic and apoptotic mechanisms. The infiltrate of the inflammatory process was composed primarily of lymphocytes and neutrophils with scattered macrophages and some plasma cells.

The treatment device 104 may efficiently and quickly cool the subcutaneous lipid-rich cells or tissue in the treatment region 136 without significantly affecting the overall body temperature of the subject 101. In general, the subject 101 has a body temperature of about 37° C. Blood circulation is one mechanism for maintaining a constant body temperature. As a result, blood flowing through the dermis and subcutaneous layer of the region acts as a heat source that counteracts the herein described cooling of the subdermal fat. Thus, providing a burst or transient of direct cooling to the subcutaneous lipid-rich cells or tissue can avoid excessive heat loss from the dermis and epidermis because it takes time for the body to respond to such cooling.

FIG. 2B is a side cross-sectional view of another specific embodiment of the treatment device 104 suitable for use in the system 100 of FIG. 1. In this embodiment, several components of the treatment device 104 are similar to those described above with reference to FIG. 2A. As such, like reference symbols refer to like features and components in FIGS. 2A and 2B.

As shown in FIG. 2B, the treatment device 104 may include an evacuation chamber 131 and one or a plurality of probes 130 (only one is shown in FIG. 2B) in fluid communication with the fluid supply 106 via the fluid lines 108a-b. The evacuation chamber 131 may include structural features that allow the probe 130 to extend into tissue to be treated after it has been drawn inside the evacuation chamber 131. For example, in the illustrated embodiment, the evacuation chamber 131 includes a first side wall 133a opposite a second side wall 133b and a top wall 135 between the first and second side walls 133a-b. The second side wall 133b includes an integrated aperture 143 configured to allow the probe 130 to pass into the evacuation chamber 131. In other embodiments, other components of the evacuation chamber 131 may include integrated apertures to allow additional probes 130 to extend through. A template, such as template 140, which may be configured to be interchangeable with other templates having differing numbers of apertures in different configurations and/or sizes, may be incorporated into one or more walls of the evacuation chamber 131.

In general, regardless of whether disposed in a template or a wall of the evacuation chamber 131, the apertures of the present invention may have different geometries to accommodate a particular probe cross-section (circular, triangular, etc.), differing diameters or opening sizes, and different orientations relative to one another when more than one aperture is used. In addition, the angle of the walls forming the apertures relative to the surface of the template 140 or the walls of the evacuation chamber 131 may vary from about 90 degrees to about 20 degrees or less to facilitate placement of probes 130 in the tissue in a desired manner.

Turning back to FIG. 2B, in further embodiments, instead of having discrete wall portions, the evacuation chamber 131 may include a generally continuous and curved wall portion, as disclosed in U.S. patent application Ser. No. 11/750,953, entitled “Method of Enhancing Removal of Heat from Subcutaneous Lipid-Rich Cells and Treatment Apparatus Having an Actuator”, filed May 18, 2007, by Rosen et al., the entire disclosure of which is incorporated herein by reference.

The evacuation chamber 131 may be configured to provide a particular volume (e.g., a rectangular, cubic, spherical, elliptical, cylindrical, etc.) into which tissue to be treated may be drawn and in different sizes to accommodate different volumes of tissue. For example, submental subcutaneous lipid-rich tissue, does not typically contain the same volume of subcutaneous lipid-rich tissue found in the abdominal or upper thigh regions. Variations in tissue volume may also vary from subject to subject.

The evacuation chamber 131 may also include a vacuum port 129 in fluid communication with a vacuum source (e.g., a vacuum pump, not shown) via a conduit 127. In the illustrated embodiment, the vacuum port 129 is positioned on the second side wall 133b. In other embodiments, the vacuum port 129 can be positioned on the first side wall 133a, the top wall 135, and/or other locations of the evacuation chamber 131. When used with the embodiment of FIG. 2B, the one or more apertures 143 may be configured to preserve the vacuum in evacuation chamber 131 regardless of the presence or absence of a probe 130. This ensures that the tissue through which the probe 130 is designed to pass is maintained in the proper position in the evacuation chamber 131 during treatment. For instance, the template 140 or the second side wall 133b containing the aperture 143 may incorporate a radially expandable valve, a reed valve, and/or any other suitable valve, to maintain an adequate seal around probe 130 prior to probe insertion, during passage of the probe 130 through the aperture, and while probe 130 is positioned within the aperture 143.

In certain embodiments, the evacuation chamber 131 may optionally include a heat exchanger. For example, as illustrated in FIG. 2B, the evacuation chamber 131 includes a thermoelectric module 137 positioned proximate to the first wall 133a. In other embodiments, additional thermoelectric modules 137 may be positioned in other parts of the evacuation chamber 131 as desired. In further embodiments, the first side wall 133a, the second side wall 133b, and/or the top wall 135 may be constructed with a thermoelectric module 137.

In further embodiments, the top wall 135 may be constructed from glass, plastic, and/or other at least partially transparent materials. In these embodiments, the treatment device 104 may optionally include a detector 105 (e.g., an ultrasound transducer) and/or a treatment applicator 141 proximate to the top wall 135. The treatment applicator 141 may include an electrical applicator (e.g., a radio frequency transducer), an optical applicator (e.g., a laser), a mechanical applicator (e.g., a high intensity focused ultrasound transducer), and/or other suitable treatment components. If an optional detector 105 and/or treatment applicator 141 is used, evacuation chamber 131 may comprise a suitable aperture to permit or facilitate transmission of energy therethrough. For instance, all or a portion of the top wall 135 may comprise silicone to permit transmission of acoustic energy when an ultrasound transducer 105 is used to monitor treatment.

In operation, a operator may place the evacuation chamber 131 at least proximate to the skin 138 of the patient 101. The operator may activate the vacuum source and withdraw air from the evacuation chamber 131 via the vacuum port 129. As air flows out of the evacuation chamber 131, a vacuum is created in the evacuation chamber 131. The vacuum may then urge a portion of the skin 138 and corresponding subcutaneous layer 128 of the subject 101 into the evacuation chamber 131. By controlling the vacuum, the operator may form a treatment region 136 that generally conforms to the evacuation chamber 131.

The operator may then insert the probe 130 through the aperture 143 and optionally, with the aid of the template 140, into the subcutaneous lipid-rich cells or tissue of the treatment region 136 by piercing the skin 138. During insertion, the operator may use imaging from the detector 105 to monitor the current position of the probe 130 and adjust the placement of the cooling elements 134 accordingly. The operator may then use the inserted probe 130 to cool the subcutaneous lipid-rich cells or tissue proximate to the cooling element 134 to form the treatment zone 142, as described above with reference to FIG. 2A.

During cooling, the operator may optionally monitor the cooling process using the detector 105. For example, the operator may monitor the growth of the treatment zone 142 based on data collected from the detector 105. The operator may also apply additional treatment to the skin 138 using the treatment applicator 141. For example, the operator may mitigate discomfort caused by the cooling and/or to protect the dermis from freezing damage by applying heat from the treatment device 141 via the top wall 135.

Optionally, the operator may pre-cool or pre-heat the treatment region 136 using the thermoelectric module 137 prior to inserting the probe 130. For example, the operator may apply a suitable voltage to the thermoelectric module 137 to cool the treatment region 136 to a temperature of about 30° C., preferably 20° C., and more preferably 10° C. before insertion. Pre-cooling the treatment region 136 may provide an anesthetic effect and/or to affect a larger area in the subcutaneous layer 128 than the treatment region 136.

The operator may efficiently achieve a desired aesthetic outcome and/or subcutaneous fat layer reduction for, e.g., body contouring and/or body sculpting using the treatment device 104. Typically, certain regions of the subject 101 have contours and/or other structural complexities that prevent proper placement of the treatment device 104. Thus, having the treatment region 136 generally conform to the evacuation chamber 131 may create a generally uniform volume that allows the operator to efficiently plan the treatment profile and place one or more probes 130.

The treatment device 104 may also enhance cooling the subcutaneous lipid-rich cells or tissue in the treatment region 136 without significantly affecting the overall body temperature of the subject 101. As described above with reference to FIG. 2A, blood flowing through the dermis and the subcutaneous layer of the treatment region acts as a heat source that counteracts the cooling of the subdermal fat. We have recognized that by compressing the treatment region 136 with the evacuation chamber 131, blood flow to the treatment region 136 may be reduced to enhance cooling the subdermal fat. We also have recognized that urging the treatment region 136 into the evacuation chamber 131 separates the subcutaneous layer 128 in the treatment region 136 from underlying and warmer muscle tissue of the subject 101, thereby additionally providing for a more efficient cooling of the targeted subcutaneous lipid-rich cells or tissue in the treatment region.

Even though the above description discloses using vacuum to form a generally uniform volume of tissue in the treatment region 136, in other embodiments, other mechanisms may also be used to create the generally uniform volume of tissue. For example, the operator may create a vacuum by burning a fuel (e.g., methanol) in the evacuation chamber 131 and quickly placing the evacuation chamber 131 onto the skin 138 of the subject 101. In other examples, compression may be used to form the generally uniform volume of tissue in lieu of vacuum.

FIGS. 3A and 3B are side elevation views partially illustrating embodiments of a probe 130 in accordance with an embodiment of the invention suitable for use as the probe 130 of FIG. 2A and FIG. 2B. Probe 130 may include a base portion 132 and a needle portion 164 extending from the base portion 132. The base portion 132 may extend along a first axis 146, and the needle portion 164 may extend along a second axis 148. In the illustrated embodiment of FIG. 3A, the needle portion 164 extends generally parallel to the base portion 132. In the illustrated embodiment of FIG. 3B, the needle portion 164 is canted relative to the base portion 132 such that the first axis 146 and the second axis 148 form an angle 149. The angle 149 may be any angle, such as between 0° and 90°. In other embodiments, the base portion 132 and the needle portion 164 may extend generally co-axially, as illustrated in FIG. 3A.

FIG. 3C-D are side elevation views partially illustrating another embodiment of the probe 130. As illustrated in FIG. 3C, the probe 130 may include a first needle portion 164a and a second needle portion 164b canted relative to the first needle portion 164a at a needle angle 147. During insertion, the second needle portion 164b may be generally parallel to the skin 138 while the base portion 132 and the first needle portion 164a are canted relative to the skin 138 at an entry angle 145. As illustrated in FIG. 3D, the entry angle 145 generally equals to 180° minus the needle angle 147. As a result, as the needle angle 147 decreases, the entry angle 145 increases.

One expected advantage of using the probe 130 is the ease of positioning the probe 130 in the treatment region to have a low insertion angle relative to the skin of the subject 101. Because the needle portion 164 is canted relative to the base portion 132, the entry angle 145 for the base portion 132 can be greater than that of the second needle portion 164b. As a result, the operator has more room to manipulate the base portion 132 when inserting the probe 130. Accordingly, the operator may more easily place the needle portion 164 generally parallel to the skin of the subject 101.

FIG. 3E is a perspective view partially illustrating another embodiment of the probe 130. In the illustrated embodiment, the probe 130 includes a shaft portion 132, a first needle portion 164a and a second needle portion 164b. As shown in FIG. 3E, the second needle portion 164b includes a plurality of needles entering through a single entry site. The second needle portions 134b may include a predetermined angle relative to the first needle portion 164a such that upon insertion into the subject's skin, the second needle portions 134b may expand into the subcutaneous tissue in a predetermined configuration. The second needle portions 134b may include a plurality of similarly angled needles relative to the first needle portion 164a as shown in FIG. 3E or may alternatively include needles with different angles relative to the first needle portion 164a, or a combination of similarly angled needles and differently angled needles.

Prior to insertion into the subject's skin, the needle portions 134a, 134b may be retracted into the shaft portion 132 in a stored position. Upon insertion, the retractable needle portions 134a, 134b may be commanded to expand into the subcutaneous lipid-rich cells or tissue of the subject 101. One expected advantage of the probe 130 is that a larger area of subcutaneous lipid-rich cells or tissue can be treated through a single entry site into the subject 101.

In any of the embodiments illustrated in FIGS. 3A-E, the needle portion 164 can incorporate a shape memory alloy including, for example, nitinol or other shape memory alloys. One expected advantage of incorporating a shape memory alloy is that the shape of the needle portion 164 can be maintained because the shape memory alloy can return the needle portion 164 to its original shape when any external stress is removed. Another expected advantage of incorporating a shape memory allow is that a treatment region shape can be predetermined to increase the efficiency and efficacy of the treatment.

To ensure that the probe 130 is inserted into the tissue at a desired depth, an optional conduit (not shown) of a fixed or adjustable length may be affixed to the template 140 or a wall of the evacuation chamber 131 (FIG. 2B) in alignment with the aperture 143. During treatment, as the needle portion 162 of the probe 130 passes through the conduit and the aperture 143 into the tissue, the base portion 132 eventually comes into contact with the free end of the conduit, thus preventing further insertion of the probe 130 into the tissue. An adjustable collar with a diameter larger than the aperture 143 may be adjustably or permanently affixed (via a detent mechanism or the like) on the probe 130 distal of handle 132 to similarly limit the travel of the probe 130 into the tissue being treated. Alternatively, an automated probe insertion mechanism similar to those used for obtaining tissue biopsies, having an adjustable dial or other mechanism for selecting the length of probe travel, may be used.

FIG. 4 is a side cross-sectional view illustrating a needle portion 164 in accordance with an embodiment of the invention suitable for use in the probe 130 shown in FIGS. 2A-2B and FIG. 3. The needle portion 164 may include an insulated section 150 and a heat exchanging section 152. The insulated section 150 may include a sleeve 151 enclosing a first conduit 156, a second conduit 158, and a chamber 153 separating the first and second conduits 156, 158 from the sleeve 151. The sleeve 151 may have a generally cylindrical shape with two closed ends or may have other suitable shapes. The chamber 153 may contain an insulating material or gas including, for example, fiberglass, silicate, air, argon, or other insulators. Alternatively, the chamber 153 may be empty.

The first and second conduits 156, 158 may be connected to the fluid lines 108a-b (shown in FIG. 2A), respectively, and extend from the insulated section 150 to the heat exchanging section 152. The heat exchanging section 152 may include a chamber 154 in fluid communication with both the first and second conduits 156, 158. The chamber 154 may be surrounded by a housing 155 extending from the sleeve 151. The housing 155 may be constructed from a thermally conductive material such as a metal, a metal alloy, or other suitable conductive materials. Optionally, the needle portion 164 also may include a sensor 162 (e.g., a temperature sensor) proximate to the housing 155. The sensor 162 may be connected to the processor 114 (shown in FIG. 1) for monitoring a process parameter (e.g., a temperature) of the subcutaneous lipid-rich cells or tissue proximate to the needle portion 164.

In operation, a fluid may be circulated to exchange heat with the subcutaneous lipid-rich cells or tissue proximate to the needle portion 164. During operation, fluid flows through the first conduit 156, the chamber 154, and the second conduit 158. The fluid in the chamber exchanges heat with the subcutaneous lipid-rich cells or tissue in the treatment zone 142 via the housing 155. The chamber 153 inhibits the fluid from exchanging heat with any surrounding tissues. The relative dimensions of the insulated section 150 and the heat exchanging section 152 may be adjusted based on the particular application.

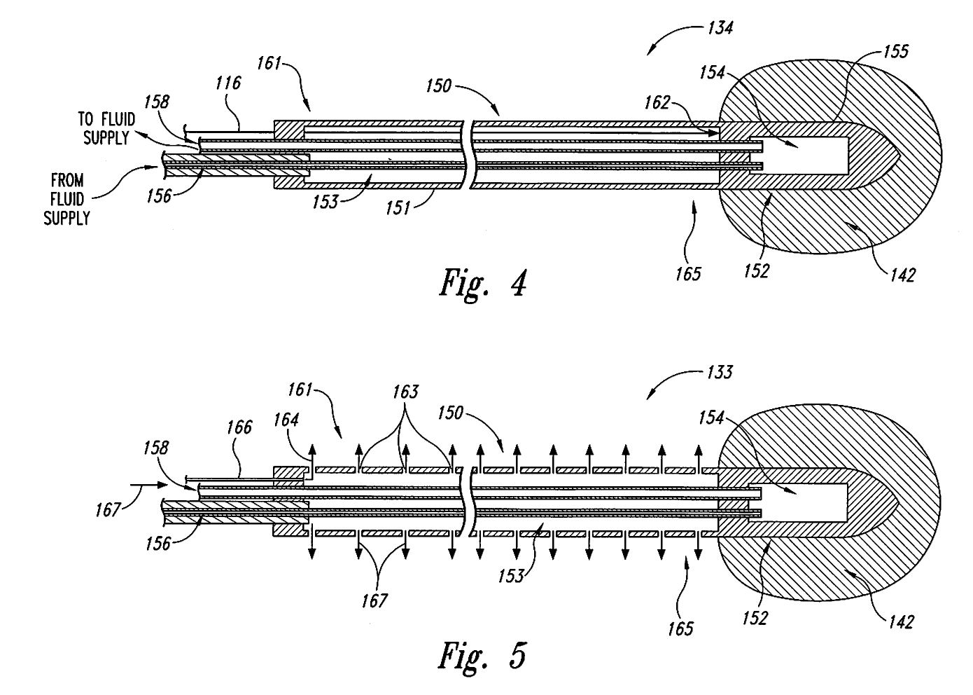

FIG. 5 is a side cross-sectional view illustrating a needle portion 133 in accordance with another embodiment of the invention suitable for use in the probe 130 shown in FIG. 2A. In this embodiment, several components of the needle portion 133 are similar to those of the needle portion 164 described above with reference to FIG. 4. As such, like reference symbols refer to like features and components in FIGS. 3 and 4. In this embodiment, the needle portion 133 includes a sleeve 151 having perforations 163 and a third conduit 166 in fluid communication with the volume 153 and the perforations 163.

The perforations 163 may identically have the same dimension or may be sized differently. For example, perforations 163 may have a progressively larger dimension (e.g., a diameter if in the form of a circle) towards the distal end 165 of the sleeve 151 from the proximal end 161 of the sleeve 151 to compensate for any pressure loss along the length of the sleeve 151 and so to ensure uniform perfusion of fluid therethrough. Other designs in which selectively non-uniform fluid dispersion through perforations 163 may also be used in connection with the present invention.

In operation, a biocompatible fluid flows into the volume 153 via the third conduit 166. The fluid flows through the perforations 163 and flushes the subcutaneous lipid-rich cells or tissue proximate to the needle portion 133 (shown by arrows 167). The fluid can be at a temperature higher than that of the cooled subcutaneous lipid-rich cells or tissue of the subject 101. Any biocompatible fluid useful for flushing the subcutaneous lipid-rich cells or tissue may be used, including, for example, saline, a tumescent fluid, a dye, therapeutic agents (e.g., antibiotic agents or anti-cancer agents, etc.), or any combination thereof.

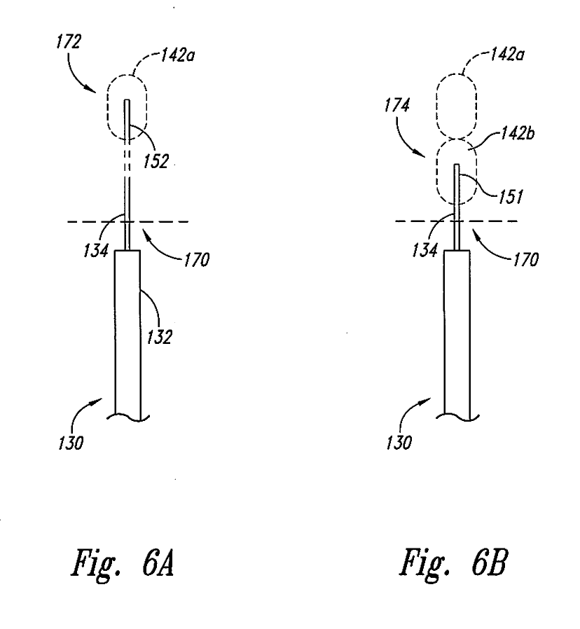

FIGS. 6A-B are top views illustrating a probe (e.g., the probe 130 of FIG. 2A) operated in accordance with another embodiment of the invention. As illustrated in FIG. 6A, the needle portion 164 is inserted at an insertion point 170 and positioned at a first location 172 within the patient. A coolant is circulated through the probe 130 such that the subcutaneous lipid-rich cells or tissue around the heat exchanging section 152 of the needle portion 164 are frozen to create a first treatment zone 142a. The circulation of the coolant is stopped after the subcutaneous lipid-rich cells or tissue in the first treatment zone 142a are frozen, and the needle portion 164 is warmed so that the heat exchanging section 152 may be removable from the first treatment zone 142a. The needle portion 164 can then be safely withdrawn from the first treatment zone 142a to a second location 174 within the patient shown in FIG. 6B. The coolant flow may be resumed to create a second treatment zone 142b. The first and second treatment zones 142a-b may be separated from each other or may form a contiguous volume of frozen tissue and may form any geometric treatment zone shape, such as planar, spherical, cubic, conical and/or any combination of these.

One expected advantage of this process is that the freezing and withdrawing steps may be repeated to create a volume of frozen subcutaneous lipid-rich cells or tissue with a single entry wound. In one embodiment, the volume of frozen subcutaneous lipid-rich cells or tissue may have an axis that is generally parallel to the skin of the subject 101. In another embodiment, the axis of the frozen volume may be canted relative to the skin. In other embodiments, the volume of frozen volume may be generally uniform in thickness or may have a varying thickness along its length.

Even though FIGS. 6A-B illustrates that the treatment zones 142 are frozen during treatment, the probe described above can be used to cool without freezing, or to cool in combination with freezing, the subcutaneous lipid-rich cells or tissue in the treatment zones 142. For example, the operator may use the probe to create a cooling area in the treatment zones 142 without freezing. In another example, the operator may use the probe to freeze a portion of the treatment zones 142 and cool another portion of the treatment zones 142 via the frozen portion.

### D. Computing System Software Modules

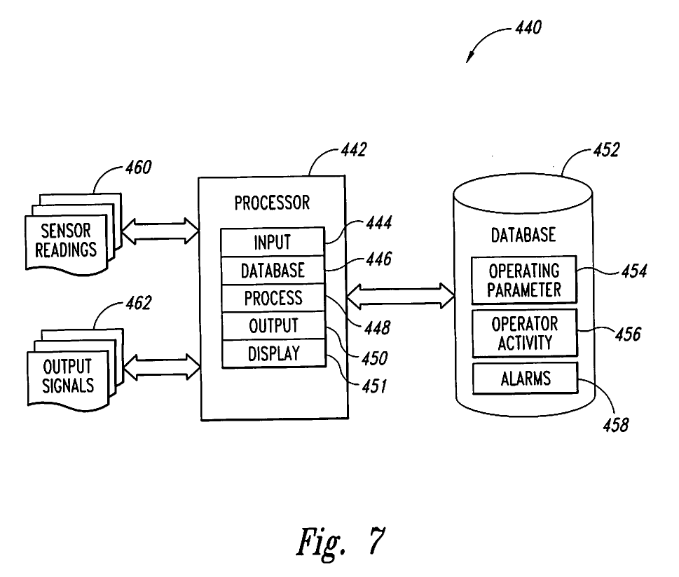

FIG. 7 illustrates a functional diagram showing exemplary software modules 440 suitable for use in the processing unit 114. Each component may be a computer program, procedure, or process written as source code in a conventional programming language, such as the C++ programming language, and may be presented for execution by the CPU of processor 442. The various implementations of the source code and object and byte codes may be stored on a computer-readable storage medium or embodied on a transmission medium in a carrier wave. The modules of processor 442 may include an input module 444, a database module 446, a process module 448, an output module 450, and optionally, a display module 451. In another embodiment, the software modules 440 may be presented for execution by the CPU of a network server in a distributed computing scheme.

In operation, the input module 444 accepts an operator input, such as process setpoint and control selections, and communicates the accepted information or selections to other components for further processing. The database module 446 organizes records, including operating parameters 454, operator activities 456, and alarms 458, and facilitates storing and retrieving of these records to and from a database 452. Any type of database organization may be utilized, including a flat file system, hierarchical database, relational database, or distributed database, such as provided by a database vendor such as Oracle Corporation, Redwood Shores, Calif.

The process module 448 may generate control variables based on the sensor readings 456, and the output module 450 generates output signals 458 based on the control variables. For example, the output module 450 may convert the generated control variables from the process module 448 into output signals 458 suitable for a direct current voltage modulator. The processor 442 optionally may include the display module 451 for displaying, printing, or downloading the sensor readings 456 and output 458 via devices such as the output device 120. A suitable display module 451 may be a video driver that enables the processor 442 to display the sensor readings 456 on the output device 120.

In certain embodiments, the process module 448 may also generate a cooling profile for the treatment region. The process module 448 may accept user inputs that define the treatment region. The user inputs may include dimensions, heat capacity, heat conductance, number of probes, coolant characteristics (e.g., temperature, flow rate, etc.), flush fluid composition, flow rate, volume and timing of perfusion, and other parameters of the treatment region. Based on these parameters, the process module 448 may calculate the cooling profile according to general heat transfer principles. For example, the process module 448 may calculate an expected cooling rate given a particular coolant temperature and flow rate. The calculated cooling profile may be used to configure the system 100 and provide the operator with expected process parameters.

In other embodiments, the measured and/or generated process parameters can be stored in a non-volatile memory (not shown) disposed in the needle portion 164 (shown in FIGS. 2-6) of the probe 130. The non-volatile memory can be configured for storing a variety of parameters (e.g., physiological measurements, operating parameters, etc.), enforcing single-patient-use with a timer and/or a counter for tracking the number of cooling/heating cycles, and/or encrypting transmitted information. The non-volatile memory can include a flash memory device (e.g., EPROM), a hard drive, an optical disk drive, or other suitable non-volatile memory devices.

### E. Methods for Controlling Cooling Subcutaneous Lipid-rich Cells or Tissue

FIG. 8 is a flow chart illustrating a method 800 of operating the process module 448 of FIG. 7 for treatment planning in accordance with an embodiment of the invention. The method 800 of FIG. 8 can be implemented as a conventional computer program for execution by the processor 442 of FIG. 7.

One embodiment of the method 800 includes stage 802 in which data of a region of the subject 101 (FIG. 1) are acquired. The data may be in graphical, numerical, text, or other form. The data may be acquired using a temperature sensor, a pressure sensor, an ultrasound transducer, a computed tomography scanner, a radioscopy scanner, an X-ray machine, an MRI scanner, and/or other suitable detector to differentiate epidermis, dermis, subdermal fat, and muscle tissue of the subject 101.

The method 800 may also include stage 804 in which the acquired data are displayed or rendered to an operator at the output device 120 (FIG. 1). In one embodiment, the image data may be displayed to the operator as a two-dimensional image profile. In other embodiments, the data may be displayed to the operator as a text listing, a three-dimensional profile, and/or using other suitable format.

After displaying the acquired data, the method 800 may continue to stage 806 in which the operator selects a desired treatment region relating to the displayed image data. In one embodiment, the operator is allowed to draw the treatment region on the displayed image data using a pointing device (e.g., a mouse, stylus, etc.). In other embodiments, the operator may enter boundary coordinates of the desired treatment region. Automated data differentiation techniques also may be used to analyze the acquired data and isolate the desired treatment region. In a still further embodiment, visual observation and/or palpation of the subject's skin and tissue in the region to be treated may be correlated or registered with the acquired data to effect stage 806.

The method 800 may then include analyzing the received treatment region to generate at least one suggested treatment regime. In one embodiment, analyzing the received treatment region may include calculating a physical dimension of the treatment region based on, e.g., the boundary coordinates of the selected region. Then, a number of required treatments may be determined by, e.g., the volume of the selected region.

In another embodiment, analyzing the received treatment region may also include calculating a number of required probes and the suggested placement of these probes based on, e.g., a rule requiring certain separation between adjacent probes. For example, an iterative procedure may be implemented to calculate the separation between a number of probes until the calculated separation is below a threshold according to the rule.

In a further embodiment, analyzing the received treatment region may also include calculating an expected cooling rate and/or a temperature profile of the treatment region based on the number of probes and their placement. The method 800 may proceed by determining whether the cooling rate exceeds a cooling threshold and/or whether an expected dermis/subdermal temperature exceeds a temperature threshold. In certain embodiments, the cooling rate and/or the temperature profile may also be calculated based on an operator-entered parameter (e.g., a number of probes).

After analyzing the received treatment region, the method 800 may continue to stage 810 in which the analysis results are provided to the operator as the suggested treatment regime. For example, the suggested treatment regime may include a depiction of the treatment region showing placement of the suggested number of probes overlaid on the displayed data, the suggested cooling rate, and/or the suggested number of treatments. A determination is made at stage 812 to decide whether the process should be continued. If the process is continued (e.g., when the operator desires to repeat the analysis or to analyze another region), the process reverts to stage 802; otherwise, the process ends.

Method 800 optionally may include one or more stages in which an image is generated showing the treatment region after a treatment is completed. Such an image or other form of data may show, for example, an ultrasonic image of the treated region, depicting the zone of frozen and/or affected tissue and overlaid with or compared against the image of the treatment region generated in stage 806. Another optional stage may include an image or other data depicting how the expected reduction of subcutaneous lipid-rich tissue or other tissue treated by the methods described herein may resolve in terms of a cosmetic effect. Such a stage may produce one or more computer-generated images, for example, projecting how the subject's body might look a number of days, weeks, or months after treatment. This projection may be based on a model that calculates the expected reduction of the lipid-rich or other tissue for a given set of treatment parameters. The projection may also be based on empirical data acquired from previous treatments on subjects of the same sex and similar body type, etc., who were treated in the same body region. These images or data may be compared to images or data of the subject's body in the treated region acquired before treatment to project the efficacy of the treatments described herein.

The method 800 may provide convenient planning for a treatment session. By using the method 800, the operator may determine the number of probes required and a cooling rate for these probes before the treatment. The method 800 may also reduce the risk of damaging the dermis and/or epidermis of the subject 101 by calculating a temperature profile of the treatment region based on a suggested or a user-entered cooling rate.

Other methods and devices as described in U.S. Pat. Nos. 6,139,544 to Mikus et al., 6,643,535 to Damasco et al., 6,694,170 to Mikus et al., U.S. Patent Application Publication Nos. US 2007/0239150, filed Sep. 7, 2005, and US 2002/0198518 to Mikus et al., filed Apr. 11, 2002, incorporated herein by reference in their entirety, may also be used to facilitate treatment and treatment planning for the methods and devices described herein.

Unless the context clearly requires otherwise, throughout the description and the claims, the words “comprise,” “comprising,” and the like are to be construed in an inclusive sense as opposed to an exclusive or exhaustive sense; that is to say, in a sense of “including, but not limited to.” Words using the singular or plural number also include the plural or singular number respectively. When the claims use the word “or” in reference to a list of two or more items, that word covers all of the following interpretations of the word: any of the items in the list, all of the items in the list, and any combination of the items in the list.

The above detailed descriptions of embodiments of the invention are not intended to be exhaustive or to limit the invention to the precise form disclosed above. While specific embodiments of, and examples for, the invention are described above for illustrative purposes, various equivalent modifications are possible within the scope of the invention, as those skilled in the relevant art will recognize. For example, while steps are presented in a given order, alternative embodiments may perform steps in a different order. The various embodiments described herein can be combined to provide further embodiments.

In general, the terms used in the following claims should not be construed to limit the invention to the specific embodiments disclosed in the specification, unless the above detailed description explicitly defines such terms. While certain aspects of the invention are presented below in certain claim forms, we contemplate the various aspects of the invention in any number of claim forms. Accordingly, we reserve the right to add additional claims after filing the application to pursue such additional claim forms for other aspects of the invention.

1. A system for freezing subcutaneous lipid-rich cells or tissue of a subject, comprising:
a fluid supply; and
a probe having internal passageways in fluid communication with the fluid supply, the probe being configured to be positioned subcutaneously at least proximate to the subcutaneous lipid rich cells.

2. The system of claim 1, further comprising a cooling device configured to non-invasively cool the subcutaneous lipid-rich cells or tissue.

3. The system of claim 2 wherein the cooling device includes an external cooling device having Peltier elements.

4. The system of claim 1, further comprising:
a temperature sensor for monitoring a temperature of the subject; and
a controller operably coupled to the temperature sensor for controlling the fluid flow based on the monitored temperature.

5. The system of claim 1 wherein the probe further comprises a needle portion canted relative to a base portion.

6. The system of claim 1, further comprising a detector selected from the group consisting of an ultrasound sensor, a computed tomography scanner, a radioscopy scanner, an X-ray machine, and an MRI scanner.

7. The system of claim 1, further comprising a plurality of probes arranged in an array, the plurality of probes being in fluid communication with the fluid supply.

8. The system of claim 7, further comprising a template for arranging the plurality of probes into the array.

9. The system of claim 1 wherein the fluid supply includes a subcooled liquid nitrogen.

10. A system for reducing subcutaneous lipid-rich cells or tissue of a subject, comprising:
a fluid supply;
a chamber having an aperture; and
a probe having internal passageways in fluid communication with the fluid supply, the probe being configured to be positioned subcutaneously at least proximate to the subcutaneous lipid rich cells through the aperture of the chamber.

11. The system of claim 10 wherein the chamber includes a heat exchanger configured to non-invasively heat or cool the subcutaneous lipid-rich cells or tissue.

12. The system of claim 10 wherein the chamber includes a wall constructed from a material that is at least partially transparent, and wherein the system further includes a detector positioned proximate to the wall of the chamber, the detector being selected from the group consisting of an ultrasound sensor, a computed tomography scanner, a radioscopy scanner, an X-ray machine, and an MRI scanner.

13. The system of claim 10 wherein the chamber includes a wall constructed from a material that is at least partially transparent, and wherein the system further includes a treatment applicator positioned proximate to the wall of the chamber, the treatment applicator being selected from the group consisting of a radio frequency transducer, a laser, and a high intensity focused ultrasound transducer.

14. A method for reducing subcutaneous lipid-rich cells or tissue of a subject, comprising:
configuring subcutaneous lipid-rich cells or tissue having a first shape to have a second shape different than the first shape;
inserting a probe into the subcutaneous lipid-rich cells or tissue having the second shape to be at least proximate to the subcutaneous lipid-rich cells or tissue; and
freezing at least a portion of the subcutaneous lipid-rich cells or tissue by providing a coolant to the inserted probe.

15. The method of claim 14 wherein configuring subcutaneous lipid-rich cells or tissue includes configuring the subcutaneous lipid-rich cells or tissue to have a generally uniform volume in the second shape.

16. The method of claim 14 wherein configuring subcutaneous lipid-rich cells or tissue includes urging the subcutaneous lipid-rich cells or tissue into a chamber and at least partially conforming the subcutaneous lipid-rich cells or tissue to the chamber.

17. The method of claim 15 wherein urging the subcutaneous lipid-rich cells or tissue into the chamber includes withdrawing air from the chamber to establish a vacuum in the chamber.

18. The method of claim 14 wherein the subcutaneous lipid-rich cells or tissue are first subcutaneous lipid-rich cells or tissue, and wherein the method further includes affecting second subcutaneous lipid-rich cells or tissue around the frozen first subcutaneous lipid-rich cells or tissue.

19. The method of claim 14 wherein the subcutaneous lipid-rich cells or tissue proximate to two adjacent probes are frozen to form a contiguous frozen volume of tissue.

20. The method of claim 14, further comprising preventing a skin surface of the subject proximate to the probes from freezing by using at least one of applying a warm saline solution to the skin surface, conductively heating the skin surface, blowing hot air at the skin surface, irradiating the skin surface, transmitting a radiofrequency signal to the skin surface, and heating the skin surface with microwave or ultrasound.

21. The method of claim 14, further comprising monitoring a process of freezing at least a portion of the subcutaneous lipid-rich cells or tissue.

22. A method for reducing subcutaneous lipid-rich cells or tissue of a subject, comprising:
collecting data of the subcutaneous lipid-rich cells or tissue of the subject;
displaying the collected data to a user;
accepting a treatment region from the user in relation to the collected data;
analyzing the accepted treatment region; and
deriving a treatment plan to reduce the subcutaneous lipid-rich cells or tissue of the subject corresponding to the collected data.

23. The method of claim 22 wherein deriving a treatment plan includes at least one of calculating a physical dimension of the treatment region, calculating a number of required treatments, calculating a number of probes and placement of the probes, and calculating a cooling rate and/or a temperature profile of the treatment region.

24. The method of claim 23, further comprising determining whether the calculated cooling rate exceeds a threshold.

25. The method of claim 23, further comprising displaying the number of probes and the placement of the probes overlaid on the collected data.
